(19) United States
(12) Patent Application Publication
(10) Pub. No.: US 2022/0187304 A1
(43) Pub. Date: Jun. 16, 2022

(54) A Method for Measuring a Concentration of a Biomarker-Analyte In Blood from Mammals

(71) Applicant: Redhot Diagnostics AB, Södertälje (SE)

(72) Inventors: Stefan Rehnmark, Södertälje (SE); Håkan Randahl, Södertälje (SE)

(73) Assignee: Redhot Diagnostics AB, Södertälje (SE)

(21) Appl. No.: 17/426,096

(22) PCT Filed: Jan. 20, 2020
(86) PCT No.: PCT/EP2020/051233

§ 371 Date: Jul. 27, 2021

(30) Foreign Application Priority Data
Jan. 28, 2019 (SE) 1950094-1

(51) Int. Cl. G01N 33/60; G01N 33/94

(52) U.S. Cl. CPC G01N 33/60; G01N 30/7233; G01N 33/942

(57) ABSTRACT

The present invention relates to a method for measuring a concentration of one or more biomarker in blood from a mammal comprising: —providing a kit of parts for sampling blood comprising a lancet and a capillary, a vial comprising an extraction fluid and one or more internal standard, which is a radioisotope of one or more biomarker adapted to assess a quality and a concentration of the one or more biomarker in the blood sample, —distributing the kit of parts to the mammal, —receiving the vial comprising a blood sample inserted into the vial, —analysing the sample to determine the quality of the sample and the concentration of the one or more biomarker in the blood of the mammal by centrifuging the vial and performing a direct analysis of the supernatant.

### TECHNICAL FIELD

The present invention relates to a method for measuring a concentration of one or more biomarker in blood from a mammal comprising providing a kit of parts for sampling of blood, distributing the kit to a location of the mammal, receiving the vial comprising a blood sample from the mammal inserted into the vial, analysing the blood sample to determine the quality of the sample and the concentration of the one or more biomarker in the blood of the mammal, by centrifuging the vial and performing a direct analysis of the supernatant.

### BACKGROUND

Personalizing medication or Tailor Dose Monitoring (TDM) of a patient and control for substance abuse requires frequent measuring of biomarkers in blood of a mammal, such as a patient or a drug addict. It is most convenient if the blood samples can be taken in a home environment without the need for professional health care personal. However, analyzing blood samples taken in a home environment is challenging, when sampling is performed by a layman and then transported to a laboratory for analysis. Several methods for quantification or qualification of a biomarker in a blood sample taken by a layman at home have been developed.

The most common method for blood sampling that exist today are so-called dry blood spot analysis, which means that blood drawn from a lancet stick in the finger is placed on a filter disc. In general blood, has to be placed on four different filter discs. The filters discs has to be dried to prevent the blood from spreading to surrounding objects. When the discs arrive at the laboratory, they are visually inspected to ensure that they are correctly filled with blood. Thereafter, the discs are extracted by a solvent and the eluate is analyzed. An extraction will never be 100%, and a validation has to be made to determine the amount of the compound/biomarker that sticks to the filter. There are no internal controls that can give an indication about how the filter discs have been treated during the sampling and transportation or about how much of the biomarker has been extracted. Furthermore, during the extraction, the blood sample will be diluted, which places higher demands on the analytic equipment and increases risks for errors in the analyzed results.

In all mass spectroscopy (MS), or LC-MS/MS methods, one or more internal standard are used as a control to ensure that the sample has undergone the process, from preparation of the sample to completed analysis, in a correct manner. Any differences are compensated for by the added internal standard. It would therefore be advantageous to use one or more internal standards early in the method. This would allow for a better control of the quality of the analysis.

US2016/0025709 discloses a kit that can be used for qualification of damaged DNA. The blood from a mammal is collected in a capillary that is put into a vial. The vial is analyzed in the laboratory. The vial does not comprise an internal standard(s) and therefore does not allow for direct analysis of a quality and concentration of a biomarker in blood of a mammal.

WO2017210218 discloses a device that may be used for collecting a blood sample, whereby a volume of blood is collected in a vial that contains desiccant beads to dry the sample prior to analysing the sample in a robotic sample processing system. Internal standard(s) are not used in this device and neither does a dried blood sampling allow for direct measurement of both quality and concentration of a biomarker.

U.S. Ser. No. 10/067,140 discloses a system for determining a concentration of a biomarker in a blood sample. The method comprises collecting blood, adding an internal standard, filtering the sample and then analysing the sample in a mass spectroscope (MS). A special device for filtering the blood is also disclosed. No kit comprising a vial with one or more internal standard is disclosed. Therefore, this system does not allow for direct quantification and qualification of a blood sample from a patient, whereby the blood sample has been transported from the patient's home to a laboratory.

Adaway JE, et al. Therapeutic drug monitoring and LC-MS/MS, J. Chromatogr. B. 2012, 883-884, 33-49, disclose a method for monitoring therapeutic drugs using LC-MS/MS. The article explains the difficulty of removing proteins and lipids from a blood sample prior to analysis. Many internal standards that can be used in the analysis are mentioned. There is no mention of a kit comprising a vial with one or more internal standard. Neither is explained how an LC-MS/MS method can be used for direct quantification and qualification of a blood sample from a patient, whereby the blood sample has been transported from the patient's home to a laboratory.

US2002019056 discloses a method for analysing dicarboxylic acids in a blood sample using esterification of the acid. The method can be used for diagnosing vitamin B12 deficiencies.

WO2014178787 discloses novel phophatidylalkanols that may be used as internal standards for measuring long-term alcohol consumption. There is no mention of a kit comprising a vial with one or more internal standard, such that a direct assessment of a quality and concentration of an amount of alcohol in a blood sample is possible.

There is a demand from the health care sector for methods that can be used in a home environment and that are simple and cost-effective. The methods available do not fulfill the requirements and have not penetrated the market. There are no methods on the market today that guaranties both assessment of quality of the sample as well as giving a good determination of the blood concentrations of the biomarker.

None of the known methods allow for normalization of the obtained results.

### SUMMARY

The aim of the present invention is to overcome the above-mentioned problems and to provide an improved method for determining the quality of a sample and the concentration of a biomarker in blood of a mammal.

The method comprises a method for measuring a concentration of one or more biomarker/analyte in blood from a mammal comprising:
providing a kit of parts for sampling blood, the kit of parts comprising or consisting of a lancet and a capillary adapted to sample a defined volume of blood from the mammal and a vial comprising or consisting of an extraction fluid together with one or more internal standard, wherein the one or more internal standard is a radioisotope of one or more biomarker selected from a group comprising or consisting of one or more of 2H 3H, 11C, 13C, 14C, 13N, 15N, 15O, 17O and 18O radioisotope of said biomarker, adapted to assess a quality and a concentration of the one or more biomarker in the blood sample,
distributing the kit of parts to a location of the mammal,
receiving the vial comprising a blood sample from the mammal inserted into the vial, analysing the blood sample to determine the quality of the sample and the concentration of the one or more biomarker in the blood of the mammal by centrifuging the vial and performing a direct analysis of the supernatant.

In an aspect, the one or more internal standard is a 2H 3H, 11C, 13C and or 14C radioisotope of the one or more biomarker adapted to assess quality and concentration of the one or more biomarker in the blood sample. In one aspect, the one or more internal standard is a 13C and/or 2H radioisotope of the one or more biomarker. In an aspect, the one or more internal standard is a 13C and 2H radioisotope of the one or more biomarker. One 13C and 2H radioisotope of the one biomarker may be used to increase the mass difference between the internal standard and the biomarker. This may improve the quality of the analysis. In another aspect, the one or more internal standard is a 2H radioisotope of the one or more biomarker. In an aspect, the one or more internal standard is a 13C radioisotope of the one or more biomarker. Such internal standards can be manufactured and can be used in such low concentrations that there is no risk for injury by radiation. An advantage of the new method is that the blood sampling can be performed by a layman at home. No trained health personal is needed. This saves time and money for the health care sector as well as for the person that is to be monitored. Due to the presence of the one or more internal standard, a direct control of the quality of the blood sample is possible. In the method of the invention, both quality and concentration of the one or more biomarker in the blood sample can be determined.

In one aspect, the extraction fluid is a solution adapted for use in the analysis method, such as mass spectroscopy (MS). A further advantage of the method is that there is no need for an additional extraction step prior to analysis for removal of a solvent. Direct analysis of the supernatant simplifies the method and saves costs and time for analysis. This also allows the method to be used in an automated setting. The extraction fluid is optimized for the one or more particular biomarker. In general, the one or more biomarker should be stable for 14 days at room temperature.

One or more internal standard may be one internal standard or more than one internal standard, such as two or three internal standards. More than one internal standard may be used to measure both quality and concentration of one or more biomarker. In one aspect, two or three internal standards are used to measure both quality and concentration of two or three biomarkers. This allows for example for measurement of an amount of a particular pharmaceutical active compound (API) as well as its metabolites. In one aspect, one internal standard is used to measure both quality and concentration of one biomarker. The internal standard may have each individually have one or two radioisotopes.

The analyzed results can thus be used as a basis for diagnosis and for giving the patient an adequate treatment and personalize the drug (API) prescription or to tailor dose monitoring. The volume of blood is determined by the volume of the capillary sampling device. The defined volume of blood may be from 10 to 100 μl or 25 to 75 μl, or 40 to 60 μl, or about 50 μl, or 10 μl, or 25 μl, or 50 μl. In one aspect, the defined volume of blood is 50 μl.

During analysis, the one or more internal standard in the extraction fluid can be used to normalize the analyzed results between different samples and the standard curve. This gives an immediate feedback whether the sample has been treated correctly, from sampling and extraction, through the logistics chain, to the analysis of the sample in the laboratory. Normalization saves time and costs for analysis. The method thus provides a quality assurance after direct analysis of the supernatant. This feedback is important for correct diagnosis, control of levels of the API and control of drug abuse. This is especially important for APIs, wherein the difference between therapeutic and toxic levels is relatively small and monitoring of concentration of the amount of API in blood of a patient is important to prevent adverse effect of the API.

The method of the invention is simple, accurate in quality and concentration assessment and cost effective.

In one aspect, the method comprises:
providing a kit of parts for sampling of blood, the kit of parts comprising or consisting of a lancet and a capillary adapted to sample a defined volume of blood from the mammal and a vial comprising or consisting of an extraction fluid together with one or more internal standard, that is optimized for one or more particular biomarker to stabilize it for 14 days at room temperature, wherein the one or more internal standard of the one or more biomarker is a radioisotope selected from a group comprising or consisting of one or more of 2H 3H, 11C, 13C, 14C, 13N, 15N, 15O, 17O and 18O radioisotope of said biomarker adapted to assess a quality and a concentration in the blood sample,
distributing the kit of parts to a location of the mammal,
taking a blood sample from the mammal by creating a wound with the lancet,
collecting a defined volume of the blood in the capillary,
entering the blood sample or the capillary into the vial,
receiving the vial comprising the blood sample from the mammal inserted into the vial,
optionally, extracting the fluid from the vial,
centrifuging the vial, and
analysing the supernatant to determine the quality of the sample and the concentration of the one or more biomarker/analyte in the blood of the mammal.

In an aspect, the one or more internal standard is a 2H 3H, 11C, 13C and or 14C radioisotope of the one or more biomarker. In one aspect, the one or more internal standard is a 13C and/or 2H radioisotope of the one or more biomarker. In an aspect, the one or more internal standard is a 13C and 2H radioisotope of the one or more biomarker. In another aspect, the one or more internal standard is a 2H radioisotope of the one or more biomarker. In an aspect, the one or more internal standard is a 13C radioisotope of the one or more biomarker. Such internal standards are relatively easy to manufacture and can be used in such low concentrations that there is no risk for injury by radiation.

In one aspect, one internal standard is used to measure both quality and concentration of one biomarker. In another aspect, the mammal is a human.

In a further aspect, the one or more internal standard is adapted for measuring a quality and concentration of one or more pharmaceutical active compound (API) that can be stabilized in an extraction fluid. In one aspect, the API is selected from the group comprising or consisting of antifungal API, antiviral API, immunodepression API, anticonvulsant API, antidepressant API, antibiotic API, anticancer API, vitamins, cardiovascular API, painkilling API and opiates. In one aspect, the API is selected from the group comprising or consisting of immunodepression API, anticonvulsant API, antidepressant API, anticancer API, vitamins, cardiovascular API, painkilling API and opiates. In an aspect, the API is selected from the group comprising or consisting of antidepressant API, anticancer API, vitamins, cardiovascular API, painkilling API and opiates. In a further aspect, the API is selected from the group comprising or consisting of antidepressant API, anticancer API, vitamins and cardiovascular API. In one aspect, the one or more internal standard is adapted for measuring quality and concentration of anticancer API.

In another aspect, the one or more internal standard is adapted for measuring a quality and concentration of one or more pharmaceutical active compound that can be stabilized in an extraction fluid, wherein the one or more internal standard is selected from the group comprising or consisting of tamoxifen, z-endoxifen, 4-hydroxytamoxifen, CPA-cyclophosphamide and its active metabolite PAM-hb, DOC-docetaxel, DOX-doxorubicine, PAC-paclitaxel, EPI-epirubicin, statins, steroids, such as testosterone, or vitamins, such as vitamin B or D, or compounds related to addictions, such as opioids, cocaine, heroin, fentanyl, cannabinoids, marijuana, benzodiazepines, hallucinogens and methamphetamine, codeine, ibuprofen, dihydrocodeine, morphine, dextropropxyphene napsylate, aminorex, lidocaine, iso-LSD, norcocaine, amitriptyline, pemoline, prazepam, imipramine, propafenone, pheniramine, chlorpromazine, amphetamine sulfate, lofexidine hydrochloride, clozapine, ecgonine methyl ester, diphenylhydramine, estazolam, 3,4-methylenedioxy-amphetamine, melatonin, doxepin, ketamine, mescaline, aprobarbital, buprenorphine, benzoylecgnine, trifluoperazine, methadone, ecgonine ethyl ester, midazolam, fentanyl, norketamine, chlordiazepoxide, caffeine, hydrocodone, fenfluramine, tramadol, lorazepam, phenylpropanolamine, flunitrazepam, amobarbital, flurazepam, phencyclidine (PCP), barbital, carbamazepine, vancomycin and phenobarbital.

In another aspect, the one or more internal standard is adapted for measuring a quality and concentration of one or more pharmaceutical active compound that can be stabilized in an extraction fluid, wherein the one or more internal standard is selected from the group comprising or consisting of tamoxifen, z-endoxifen and 4-hydroxytamoxifen. Especially in the case of tamoxifen it is advantageous to use more than one internal standard to also measure active metabolites of tamoxifen in the blood sample. In one aspect, the internal standards are radioisotopes of tamoxifen, z-endoxifen and 4-hydroxytamoxifen. In another aspect, the internal standards are radioisotopes of tamoxifen and z-endoxifen. In a further aspect, the internal standards are radioisotopes of tamoxifen and 4-hydroxytamoxifen. In yet another aspect, the internal standards are radioisotopes of z-endoxifen and 4-hydroxytamoxifen. In one aspect, the internal standard is a radioisotope of tamoxifen. In a further aspect, the one or more internal standard is a 13C radioisotope of tamoxifen, z-endoxifen and/or 4-hydroxytamoxifen.

Tamoxifen is a pre-drug that will be metabolized to active substances, whereas z-endoxifen and 4-OH tamoxifen are the most active metabolites. The concentration of tamoxifen is measured to find out how much drug there is available and z-endoxifen and 4-OH tamoxifen are measured to determine that the patient has blood concentrations of this anti-cancer API within the narrow therapeutic window.

In another aspect, the one or more internal standard is selected from the group comprising or consisting of one or more compounds related to addictions, such as opioids, cocaine, heroin, fentanyl, cannabinoids, marijuana, benzodiazepines, hallucinogens, methamphetamine, amphetamine, codeine, dihydrocodeine, phencyclidine (PCP), morphine, iso-LSD, norcocaine, fentanyl, methadone, hydrocodone and tramadol.

In a further aspect, the one or more internal standard is selected from the group comprising or consisting of vitamins, such as vitamin B or D. In a further aspect, the one or more internal standard is a radioisotope of methylmalonic acid adapted for measuring B-vitamin deficiency. In a further aspect, the one or more internal standard is a 2H or a 13C radioisotope of methylmalonic acid. In a further aspect, the one or more internal standard is a 2H and 13C radioisotope of methylmalonic acid. In yet a further aspect, the one or more internal standard is a radioisotope of dihydrotachysterol adapted for measuring D-vitamin deficiency. In a further aspect, the one or more internal standard is a 2H or a 13C radioisotope of dihydrotachysterol. In a further aspect, the one or more internal standard is a 2H and 13C radioisotope of dihydrotachysterol.

In another aspect, the one or more internal standard is selected from the group comprising or consisting of lorazepam, phenylpropanolamine, flunitrazepam, amobarbital, flurazepam, PCP, barbital, carbamazepine, chlordiazepoxide, aprobarbital, vancomycin and phenobarbital.

In a further aspect, the one or more internal standard is selected from the group comprising or consisting of CPA-cyclophosphamide and its active metabolite PAM-hb, or DOC-docetaxel, DOX-doxorubicine, PAC-paclitaxel and EPI-epirubicin.

In yet a further aspect, the one or more internal standard is selected from the group comprising or consisting of statins selected from the group comprising or consisting of atorvastatin, fluvastatin, cerivastatin, lovastatin, mevastatin, pitavastatin, pravastatin, rosuvastatin and simvastatin, or mixtures thereof. In an aspect, the internal standard is selected from the group comprising or consisting of pravastin, rosuvastatin, simvastatin and atorvastatin.

In yet another aspect, the one or more internal standard is selected from the group comprising or consisting of steroid hormones, such as glucocorticoids, mineralocorticoids, androgens, oestrogens and progestogens. In one aspect, the steroid hormones is selected from the group comprising or consisting of alclometasone, prednisone, dexamethasone, triamcinolone, cortisone, fludrocortisone, oxandrolone, oxabolone, testosterone, nandrolone, diethylstilbestrol (DES) and estradiol, norethisterone, medroxyprogesterone acetate, hydroxyprogesterone caproate, cyproterone acetate, mifepristone and gestrinone, or mixtures thereof. In an aspect, the internal standard is selected from the group comprising or consisting of aldosteron, androsteron, crotisol, progetsteron and testosteron.

In one aspect, the one or more internal standard is an antidepressant API selected from the group comprising or consisting of selective serotonin reuptake inhibitors, serotoning norepinephrine reuptake inhibitors, serotonin modulators and stimulators, serotonin antagonists and reuptake inhibitors, norepinephrine reuptake inhibitors, norepinephrine-dopamine reuptake inhibitors, tricyclic antidepressants, tetracyclic antidepressants, monoamine oxidase inhibitors and NMDA receptor antagonists or mixtures thereof.

In another aspect, the one or more internal standard is an antidepressant API selected from the group comprising or consisting of bupropiontrazodone, nefazodone, vilazodone, vortioxetine, mitriptyline, bupropion, bupropion, bupropion, bupropion, bupropion, citalopram, desvenlafaxine, duloxetine, escitalopram, fluoxetine, mirtazapine, nortriptyline, paroxetine, sertraline, trazodone and venlafaxine, or mixtures thereof. In an aspect, the internal standard is selected from the group comprising or consisting seratralin, citalpram, excitaopram, venlafaxin and flouxetin.

In a further aspect, the one or more internal standard is selected from the group comprising or consisting of ibuprofen, dextropropxyphene napsylate, aminorex, lidocaine, amitriptyline, pemoline, prazepam, imipramine, propafenone, pheniramine, chlorpromazine, amphetamine sulfate, lofexidine hydrochloride, clozapine, ecgonine methyl ester, diphenylhydramine, estazolam, 3,4-methylenedioxy-amphetamine, melatonin, doxepin, ketamine, mescaline, buprenorphine, benzoylecgnine, trifluoperazine, ecgonine ethyl ester, midazolam, norketamine, caffeine and fenfluramine.

In another aspect, the one or more internal standard is phosphatidylethanol 16:0/18:1 (PEth-16:0/18:1, PEth 16:0/18:2, PEth 18:1/16:0, and/or PEth 18:2/16:0)) adapted for measuring long-term alcohol consumption in a mammal, such as a human. In a further aspect, the one or more internal standard is a 2H radioisotope of phosphatidylethanol 16:0/18:1 (PEth-16:0/18:1, PEth 16:0/18:2, PEth 18:1/16:0 and/or PEth 18:2/16:0).

In a further aspect, the blood sample is analysed using mass spectrometry (MS), liquid chromatography-mass spectrometry (LC-MS, LC-MS/MS, gas chromatography (GC-MS) or GC-MS/MS. In one aspect, blood sample is analysed using mass spectrometry (MS).

In one aspect, the extraction fluid is selected from the group comprising 2-propanol, methanol and acetonitrile, formic acid, or mixtures thereof. The fluid is adapted to substantially stop all enzyme activity in the blood sample, stabilize the sample and extract the biomarker from the blood sample.

The invention also relates to a kit of part comprising or consisting of a lancet and a capillary adapted to sample a defined volume of blood from the mammal and a vial comprising or consisting of an extraction fluid together with one or more internal standard for use in a method of diagnosing a disease. In one aspect, the disease is selected from the group comprising or consisting of addiction, such as alcoholism, or a disease like cancer, such as breast cancer or prostate cancer, or cardiovascular diseases, high blood pressure, viral, fungal or bacterial infection, epilepsy, depression, pain, vitamin deficiency, immune-deficiency or steroid deficiency.

The kit can be used in the method of the invention by a layman at home. The ease of use of the kit as well as the simplicity of using the supernatant for analysis reduces the risk for errors during all steps of the method. In case of pharmaceutical active compounds, whereby frequent monitoring is important due to a narrow therapeutic window, costs for health care are reduced because no trained health care personal are needed for the monitoring. This is believed to improve compliance of the patient and thus efficacy and efficiency of the treatment. With the method of the invention there is no need for the patient to go to a health care center and there is no need for a cold chain transport to the analyzing laboratory.

In an aspect, the method or the kit is used together with an interpretation of the analysed result by a health care professional, such as a medical doctor.

In another aspect, the method or the kit as defined above is used for diagnosing a disease.

In one aspect, the method or the kit as defined above is used for diagnosing alcoholism. In another aspect, the method as defined above is used for diagnosing drug abuse.

In another aspect, the method or the kit as defined above is used for diagnosing a vitamin deficiency.

In another aspect, the method or the kit as defined above is used for monitoring an amount of API, such as tamoxifen, in blood of a patient.

In one aspect, the method or the kit as defined above is used for Tailor Dose Monitoring (TDM).

In an aspect, the method as defined above is for use in measuring or monitoring a quality and concentration of one or more pharmaceutical active compound (API) in the blood of a mammal, whereby the pharmaceutical active compound has a narrow therapeutic window selected from the group comprising or consisting of tamoxifen, digoxin, digitoxin, fosphenytoin, phenytoin, ethosuximide, alfentanil, tacrolimus, meperidine, temsirolimus, sirolimus, thiopental, fentanyl, alfentanil, theophylline, cyclosporine, clonidine, amitriptyline, protriptyline, imipramine, nortriptyline, quinidine, levothyroxine, carbamazepine, phenobarbital, ergotamine, dihydroergotamine and heparin. In another aspect the pharmaceutical active compound has a narrow therapeutic window selected from the group comprising or consisting of tamoxifen, digoxin, digitoxin, fosphenytoin, levothyroxine and carbamazepine. In another aspect the pharmaceutical active compound has a narrow therapeutic window selected from tamoxifen.

### BRIEF DESCRIPTION OF THE DRAWINGS

The invention will now be explained more closely by the description of different embodiments of the invention and with reference to the appended figures.

FIG. 1 shows an example of a kit of parts.

FIG. 2 shows a schematic overview of the method.

FIG. 3 shows a correlation between intravenous versus capillary blood sampling.

Tables 4 to 8, listing pharmaceutical active compounds and suitable internal standards.

### DETAILED DESCRIPTION

A “narrow therapeutic window” of a pharmaceutical active compound (API)/drug is defined as a small difference in dose or blood concentration between a therapeutic effective dose/concentration and a concentration that may lead to serious therapeutic failures or adverse drug reactions. Serious events are those, which are persistent, irreversible, slowly reversible, or life-threatening, possibly resulting in hospitalization, disability, or even death, or compounds having little difference between toxic and therapeutic doses of said compound. These types of compounds often need frequent monitoring of the concentration of the API in the blood of a mammal.

An “extraction fluid” means a solution comprising an analytical reference fluid with a known concentration of the internal standard. Preferably, said fluid can be directly used after centrifugation in an analytical instrument to measure the quality and concentration of the one or more biomarker.

An “internal standard” means a radioisotope of a biomarker in a known concentration of the biomarker in the extraction fluid.

An “analyte” means a molecule/biomarker/active pharmaceutical compound (API) present in the blood sample that will be analysed and quantified in the method.

A “radioisotope” means radioactive isotopes of an element, which are atoms that contain an unstable combination of neutrons and protons, or an excess energy in their nucleus. The radioactive isotope may be 13C or 2H (also written as “D” for deuterium, which is the same as the term “1H” used in the priority document). Further examples of suitable radioisotopes that may be incorporated include 3H (also written as “T” for tritium), 11C, 14C, 13N, 15N, 15O, 17O, 18O, 18F, 35S, 36Cl, 82Br, 75Br, 76Br, 77Br, 123I, 124I, 125I and 131I. The radioisotope that is used will depend on the specific application of that radio-labelled derivative. In some aspects, the radioisotope is 2H. In some embodiments, the radioisotope is 3H. In some aspects, the radioisotope is 13C. In some aspects, the radionuclide is 14C. In some aspects, the radioisotope is 11C. And in some aspects, the radioisotope is 180. The internal standard may be one or more radioactive biomarkers, whereby each biomarker may have one or more radioactive isotopes.

A “mammal” means a human or an animal, such as a horse, dog, cat, cow or pig. The human may be a patient.

The term “addiction or equivalents thereof” as used herein includes addiction as a disease.

The term “disease” as used herein is meant to include disorders, illnesses and other sicknesses.

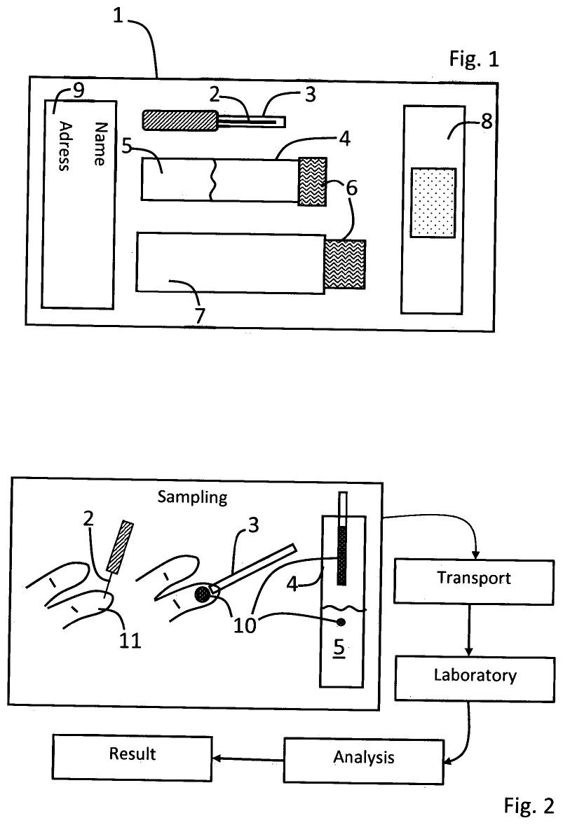

As shown in FIG. 1, the capillary kit 1 consists of a lancet 2, a capillary or capillary tube 3 that can take an exact volume of whole blood from 10 to 100 μl, or 50 μl and a vial 4 with a cap 6 comprising or consisting of an extraction fluid 5 with the one or more internal standard, a transport vial 7 with a cap 6, a plaster 8 and an address label 9.

The extraction fluid is optimized for the one or more specific biomarker, API or API metabolite to be measured or analyzed. Optimization is done so that the one or more biomarker is preferably stable for 7 or 14 days at room temperature, in the extraction fluid. In addition to a solvent, the extraction fluid contains an exact amount of one or more internal standard, that is, the one or more biomarker labeled with one or more radioisotope, such that only the mass of the internal standard differs from the biomarker/analyte. The one or more internal standard is adapted to assess a quality and concentration of the one or more biomarker in the blood sample.

The one or more internal standard is a radioisotope selected from a group comprising or consisting of one or more of 2H 3H, 11C, 13C, 14C, 13N, 15N, 15O, 17O and 18O radioisotope. The radioisotope may be a 2H 3H, 11C, 13C, 14C radioisotope. The radioisotope may be a 13C radioisotope. The radioisotope may be a 2H radioisotope. When more than one internal standard is used a combination of different radioisotopes may be used. For example, one of the internal standards may be a 2H radioisotope for one biomarker and another internal standard may be a 13C radioisotope of a different biomarker.

Tamoxifen, z-endoxifen and 4-OH tamoxifen may all be 13C labeled. If the radiolabeled molecular mass does not differ significantly from the none-radiolabeled molecular mass, the same molecule is labeled with both 13C- and 2H-radioisotopes. For methylmalonic acid (MMA) for example, the 13C MMA-radioisotopes and the 2H-MMA-radioisotopes differ only by one atom. Therefore, the same molecule may be labeled with both 13C— and 2H-MMA-radioisotopes to improve the resolution in the analyze.

To avoid interference with endogenous MMA, a standard curve is made by a labelled 13C-MMA and the internal standard is labelled both with 13C— and 2H-MMA

As shown in FIG. 2, the blood sample is taken by a stick of the lancet 2 into a finger 11 giving a wound from which 10 to 100 μL of blood 10 is collected. The blood sample is immediately transferred into the vial 4 comprising or consisting of the extraction fluid 5 and one or more internal standard and shaken vigorously. The extraction fluid stops all enzyme activities in the blood 10 and an extraction of the one or more biomarker from the blood sample to the extraction fluid occurs.

The blood sample 10 will then be transported to a laboratory for analysis. The known concentration and behavior of the internal standard(s) in the analyze will immediately indicate if the sample has not been properly handled, such as low blood volume or degradation due to heat.

The quality assurance that the internal standard(s) provides for the sample is unprecedented to any other method available on the market. Which is particularly important if the sample is taken in a home environment by a layman.

When the vial 4 with the extraction fluid containing the sample arrives at the laboratory, the vial is vortexed vigorously and centrifuged. The supernatant is transferred to a tube, which can be directly used in an e.g. LC-MS/MS instrument for analysis and concentration determination of the biomarker. The blood sample may be analysed using mass spectrometry (MS), liquid chromatography-mass spectrometry (LC-MS, LC-MS/MS, gas chromatography-mass spectrometry (GC-MS) or GC-MS/MS. The analysis can be atomized in a robotic setting.

The one or more internal standard in the extraction fluid is the same as that used in the standard curve in the analysis method. Preferably, the one or more internal standard is from the same production batch, although this is not necessary since it is possible to normalize different batches in relation to each other.

The one or more internal standard may be a biomarker for measuring a quality and concentration of any pharmaceutical active compound that can be stabilized in an extraction fluid. Examples of APIs are shown in tables 4 to 8. Suitable APIs may be selected from the group comprising tamoxifen, z-endoxifen, 4-hydroxytamoxifen, statins, steroids, such as testosterone, or vitamins, such as vitamin B or D, or compounds related to addictions, such as opioids, cocaine, heroin, fentanyl, cannabinoids, marijuana, benzodiazepines, hallucinogens and methamphetamine.

The one or more internal standard may be a biomarker for measuring a quality and concentration of a statin selected from the group comprising or consisting of atorvastatin, fluvastatin, cerivastatin, lovastatin, mevastatin, pitavastatin, pravastatin, rosuvastatin and simvastatin or mixtures thereof.

The one or more internal standard may be a biomarker for measuring a quality and concentration of a steroid hormone, such as glucocorticoids, mineralocorticoids, androgens, oestrogens and progestogens. In one aspect, the steroid hormones is selected from the group comprising or consisting of alclometasone, prednisone, dexamethasone, triamcinolone, cortisone, fludrocortisone, oxandrolone, oxabolone, testosterone, nandrolone, diethylstilbestrol (DES) and estradiol, norethisterone, medroxyprogesterone acetate, hydroxyprogesterone caproate, cyproterone acetate, mifepristone and gestrinone or mixtures thereof.

The one or more internal standard may be a biomarker for measuring a quality and concentration of an anti-depressive selective serotonin reuptake inhibitors, serotonin-norepinephrine reuptake inhibitors, serotonin modulators and stimulators, serotonin antagonists and reuptake inhibitors, norepinephrine reuptake inhibitors, norepinephrine-dopamine reuptake inhibitors, tricyclic antidepressants, tetracyclic antidepressants, monoamine oxidase inhibitors and NMDA receptor antagonists or mixtures thereof.

The anti-depressant API may be selected from the group comprising or consisting of bupropiontrazodone, nefazodone, vilazodone, vortioxetine, mitriptyline, bupropion, bupropion, bupropion, bupropion, bupropion, citalopram, desvenlafaxine, duloxetine, escitalopram, fluoxetine, mirtazapine, nortriptyline, paroxetine, sertraline, trazodone and venlafaxine, or mixtures thereof.

The one or more internal standard may be a radioisotope of tamoxifen, z-endoxifen and/or 4-hydroxytamoxifen for measuring the amount of tamoxifen in blood of a patient. The API may be tamoxifen and the internal standards may be a radioisotope of tamoxifen, z-endoxifen and/or 4-hydroxytamoxifen. The API may be tamoxifen and the internal standards may be a radioisotope of tamoxifen, z-endoxifen and 4-hydroxytamoxifen. The radioisotope(s) may be a 13C radioisotope. The radioisotope(s) may be a 2H radioisotope and 13C radioisotope.

The one or more internal standard may be a radioisotope of phosphatidylethanol 16:0/18:1, which is a biomarker for measuring long-term alcohol consumption. The radioisotope may be a 2H radioisotope.

The one or more internal standard may be a radioisotope of methylmalonic acid for measuring vitamin B insufficiency. The radioisotope may be a 2H radioisotope. The radioisotope may be a 13C radioisotope. The radioisotope may be a 2H and 13C radioisotope.

The one or more internal standard may be a radioisotope of dihydrotachysterol (Vitamin D) adapted for measuring D-vitamin deficiency. The radioisotope may be a 2H radioisotope. The radioisotope may be a 13C radioisotope. The radioisotope may be a 2H and 13C radioisotope.

The one or more internal standard may be a biomarker for measuring a quality and concentration of one or more pharmaceutical active compound having a small therapeutic window. The API may be selected from the group comprising or consisting of tamoxifen, digoxin, digitoxin, fosphenytoin, phenytoin, ethosuximide, alfentanil, tacrolimus, meperidine, temsirolimus, sirolimus, thiopental, fentanyl, alfentanil, theophylline, cyclosporine, clonidine, amitriptyline, protriptyline, imipramine, nortriptyline, quinidine, levothyroxine, carbamazepine, phenobarbital, ergotamine, dihydroergotamine and heparin. The pharmaceutical active compound that has a narrow therapeutic window may be selected from the group comprising or consisting of tamoxifen, digoxin, digitoxin, phosphenytoin, levothyroxine and carbamazepine. The pharmaceutical active compound may be tamoxifen.

In an example of the method the following step are taken;
providing a kit of parts for sampling of blood, the kit of parts comprising a lancet and a capillary adapted to sample a defined volume of blood from the human and a vial comprising an extraction fluid, which is 2-propanol or tetrahydrofuran, together with an internal standard, which is a 2H radioisotope of phosphatidylethanol 16:0/18:1,
distributing the kit of parts to a location of the mammal,
taking a blood sample from the mammal by creating a wound with the lancet,
collecting a 50 μl volume of the blood in the capillary,
entering the blood sample or the capillary into the vial,
receiving the vial comprising the blood sample from the mammal inserted into the vial,
optionally, extracting the fluid from the vial,
centrifuging the vial, and
analysing the supernatant to determine the quality of the sample and the concentration of phosphatidylethanol in the blood of the mammal.

In another example of the method the following step are taken;
providing a kit of parts for sampling of blood, the kit of parts comprising a lancet and a capillary adapted to sample a defined volume of blood from the human and a vial comprising an extraction fluid, which is acetonitrile, together with an internal standard, which is a 2H radioisotope of methylmalonic acid,
distributing the kit of parts to a location of the mammal,
taking a blood sample from the mammal by creating a wound with the lancet,
collecting a 50 μl volume of the blood in the capillary,
entering the blood sample or the capillary into the vial,
receiving the vial comprising the blood sample from the mammal inserted into the vial,
optionally, extracting the fluid from the vial,
centrifuging the vial, and
analysing the supernatant to determine the quality of the sample and the concentration of methylmalonic acid in the blood of the mammal.

In a further example of the method the following step are taken;
providing a kit of parts for sampling of blood, the kit of parts comprising a lancet and a capillary adapted to sample a defined volume of blood from the human and a vial comprising an extraction fluid, which is methanol, acetonitrile or formic acid, together with an internal standard, which is a 13C radioisotope of tamoxifen, z-endoxifen and 4-hydroxytamoxifen,
distributing the kit of parts to a location of the mammal,
taking a blood sample from the mammal by creating a wound with the lancet,
collecting a 50 μl volume of the blood in the capillary,
entering the blood sample or the capillary into the vial,
receiving the vial comprising the blood sample from the mammal inserted into the vial,
optionally, extracting the fluid from the vial,
centrifuging the vial, and
analysing the supernatant to determine the quality of the sample and the concentration of tamoxifen, z-endoxifen and 4-hydroxytamoxifen in the blood of the mammal.

In a further example of the method the following step are taken;
providing a kit of parts for sampling of blood, the kit of parts comprising a lancet and a capillary adapted to sample a defined volume of blood from the human and a vial comprising an extraction fluid, which is methanol, acetonitrile or formic acid, together with an internal standard, which is a 13C radioisotope of an antidepressant API (e.g. seratralin, citalpram, excitaopram, venlafaxin and flouxetin, or a statin (e.g. pravastin, rosuvastatin, simvastatin and atorvastatin) or a steroid hormone (e.g. aldosteron, androsteron, crotisol, progetsteron and testosteron),
distributing the kit of parts to a location of the mammal,
taking a blood sample from the mammal by creating a wound with the lancet,
collecting a 50 μl volume of the blood in the capillary,
entering the blood sample or the capillary into the vial,
receiving the vial comprising the blood sample from the mammal inserted into the vial,
optionally, extracting the fluid from the vial,
centrifuging the vial, and
analysing the supernatant to determine the quality of the sample and the concentration of said anti-depressant API, statin and steroid hormone (e.g. sertralin, atorvastatin or testosterone), in the blood of the mammal.

Centrifuging may be done at 16400 rpm. Analysis may be performed in an LC-MS/MS instrument. Other examples of biomarkers of interest are shown in table 1.

TABLE 1
Drugs

| | | |
|---|---|---|
| Codeine | Ibuprofen | Dihydrocodeine |
| Morphine | Dextropropxyphene | Aminorex |
| | napsylate | |
| Lidocaine | Iso-LSD | Norcocaine |
| Amitriptyline | Pemoline | Prazepam |
| Imipramine | Propafenone | Pheniramine |
| Chlorpromazine | Amphetamine sulfate | Lofexidine hydrochloride |
| Clozapine | Ecgonine methyl ester | Diphenylhydramine |
| Estazolam | 3,4-Methylenedioxy- | Melatonin |
| | amphetamine | |
| Doxepin | Ketamine | Mescaline |
| Aprobarbital | Buprenorphine | Benzoylecgnine |
| Trifluoperazine | Methadone | Ecgonine ethyl ester |
| Midazolam | Fentanyl | Norketamine |
| Chlordiazepoxide | Caffeine | Hydrocodone |
| Fenfluramine | Tramadol | Lorazepam |
| Phenylpropanolamine | Flunitrazepam | |
| Amobarbital | Flurazepam | PCP |
| Barbital | Carbamazepine | Phenobarbital |
| Vancomycin | | |

Further pharmaceutical active compounds may be CPA-cyclophosphamide and its active metabolite PAM-hb, or DOC-docetaxel, DOX-doxorubicine, PAC-paclitaxel, EPI-epirubicin.

The solvent used in the extraction fluid comprises an organic solvent or a combination of two or more organic solvents. The solvents may be selected from the group comprising or consisting of 2-propanol, methanol and acetonitrile, formic acid, or mixtures thereof. For the analyte PEth-16:0/18:1, the solvent may be 2-propanol or tetrahydroferan. For the analyte metylmalonic acid, the solvent may be acetonitrile. For the analyte tamoxifen, z-enoxifen, 4-hydroxytamoxifen, the solvent may be methanol, acetonitrile, formic acid, or mixtures thereof. Table 2 lists other examples of suitable solvents.

Other examples of pharmaceutical active compounds and internal standards are shown in tables 4 to 8 in the attached drawings. (Adaway J E, Therapeutic drug monitoring and LC-MS/MS, J. Chromatogr. B. 2012, 883-884, 33-49.)

TABLE 2
Solvents

| | |
|---|---|
| Methanol | Chloro-butane |
| Ethanol | Ethyl-ether |
| Isopropanol | Methyl tert-butyl ether |
| Butanol | Ethanol |
| Acetonitrile | Ethyl acetate |
| Tetrahydrofuran | Acetic acid |
| Acetone | Ammonium hydroxide |
| Dichloromethane | Ammonium acetate |
| Formic acid | |

The method and the kit of parts may be used for diagnosing a disease. The disease may be any disease related to the analyzed biomarker/analyte. Example of diseases may be any form of addiction or abuse, such as alcoholism, opiate abuse, or cancer, such as breast cancer, prostate cancer, or cardiovascular diseases, such as high blood pressure, or steroid deficiency diseases.

The method and the kit of parts may be used for Tailor Dose Monitoring (TDM).

The method and the kit of parts may be used for diagnosing and/or monitoring drug abuse, or alcoholism.

### Example 1, Method of Measuring Long Term Alcohol Use and Validation of the Method

The kit as defined above was used. The kit comprised a vial that contained 200 μl of extraction solution 80% isopropanol with 20% tetrahydrofuran and 0.1 μM D5-PEth 16:0/18:1 (i.e. 2H radioisotope of phosphatidylethanol 16:0/18:1).

50 μl blood was collected from human volunteers using the lancet and added to the extraction solution in the vial. Or 50 μl blood was collected from human volunteers using venous blood. The blood from seven different human volunteers have been collected.

The vials were transported to the laboratory for analysis.

The vials were vortexed and centrifuged. 20 μl of the supernatant was directly injected into the LC-MS/MS system. The LC-MS/MS system contains: Mobile phase A; 20% acetonitrile, 0.5 mM ammonia and 1 mM acetic acid in water, mobile phase B; 20% acetonitrile, 20% tetrahydrofuran and 60% 2-propanol were used.

The test showed that the same results were obtained independent how the blood samples were collected from the human volunteers.

The analysis allows for the quantification of low levels of 0.005 mmol/ml.

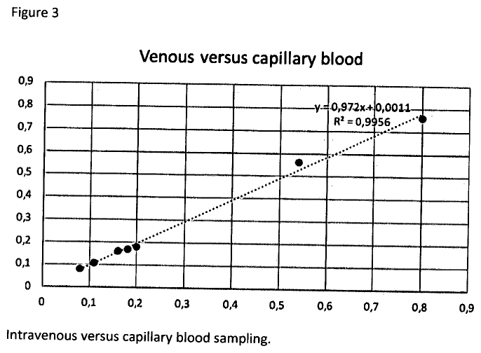

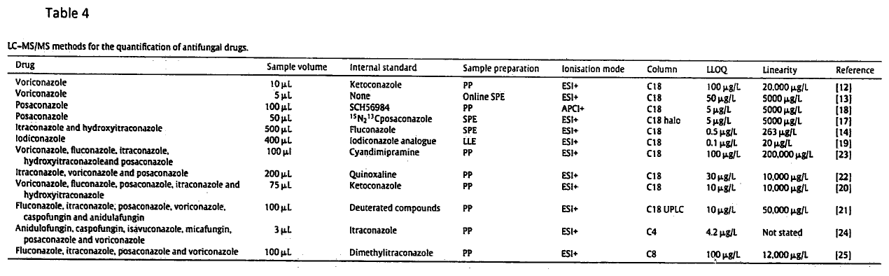

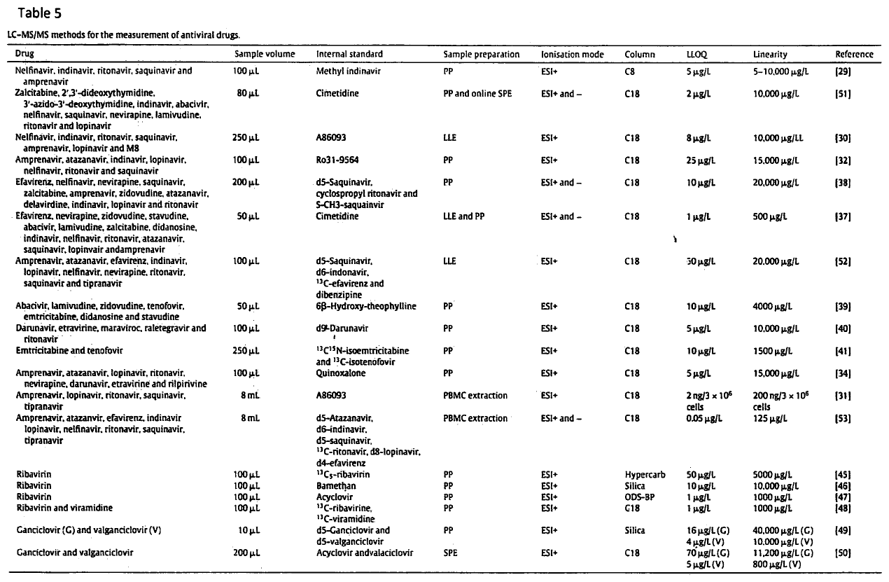

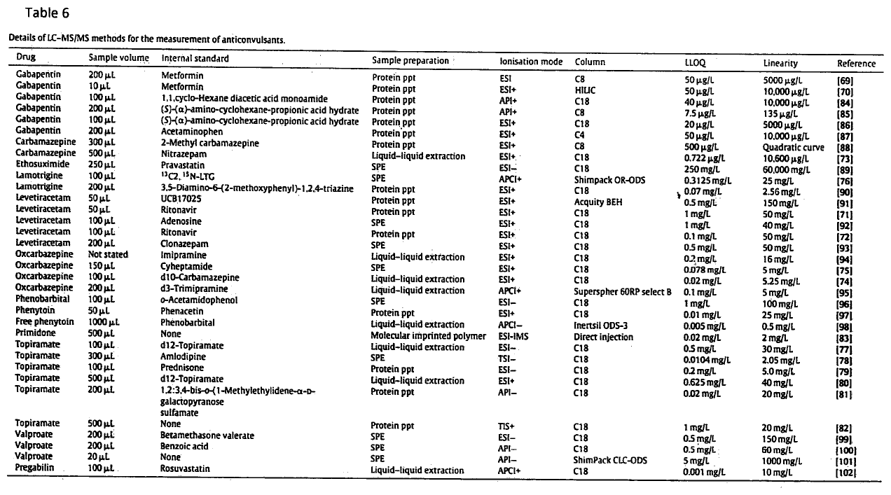

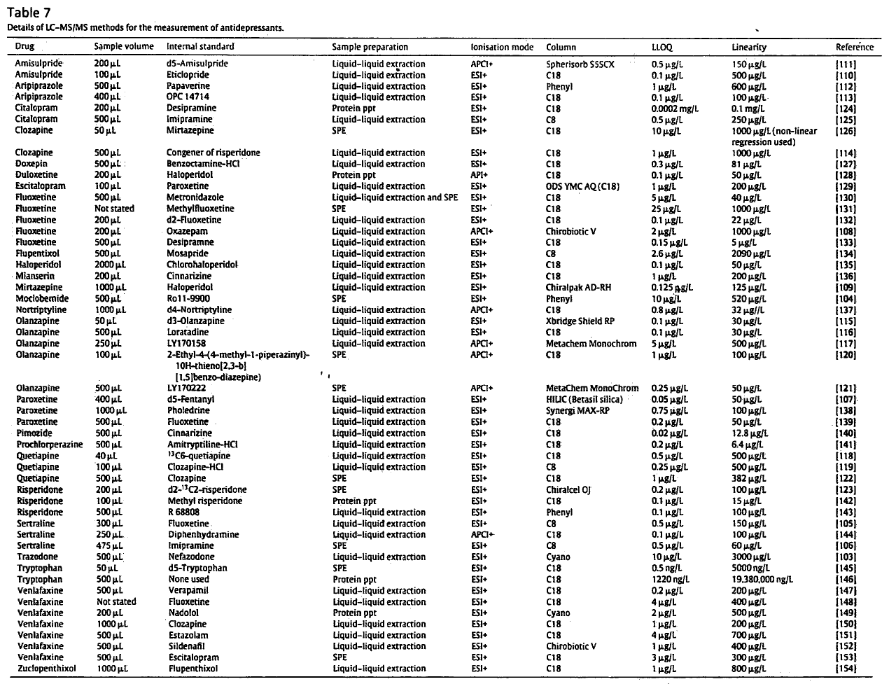

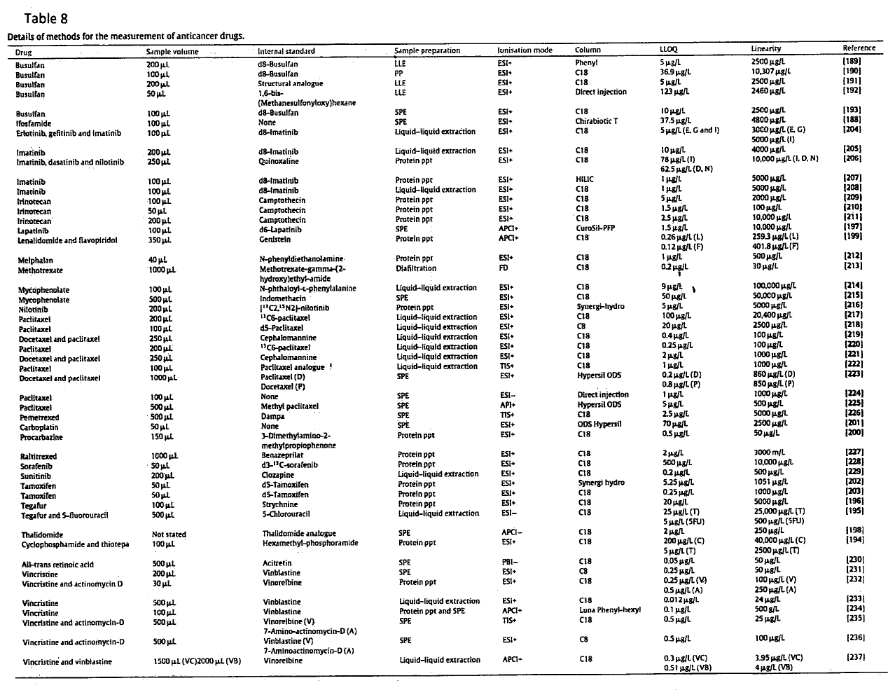

The results in table 3 and FIG. 3 show that there are no differences between a venous blood sample and a sample taken by lancet in the finger.

TABLE 3

| Individual | Conc. | % CV |
|---|---|---|
| 1 | 0.16 | 7 |
| 2 | 0.78 | 4 |
| 3 | 0.55 | 3 |
| 1 | 0.18 | 4 |
| 3 | 0.52 | 4 |
| 5 | 0.19 | 5 |
| 6 | 0.11 | 7 |
| 7 | 0.08 | 3 |
| 8 | 0.18 | 2 |

Table 1 Intravenous vs. capillary blood sampling: The concentration of triplicates from an intravenous sample and capillary blood sampled in triplicate are compared. Samples are collected from 7 healthy individuals, in a total of nine sample extractions, at two different days.

### Example 2, Monitoring Amount of Tamoxifen in Blood of a Patient

The kit as defined above was used. The kit comprised a vial that contained 150 μl extraction solution containing acetonitrile with 0.2% formic acid and 0.1 ng/mL each of 13C-labeled tamoxifen, 13C-labeled tamoxifen, 13C-labeled z-endoxifen and 13C-labeled 4-OH tamoxifen, 0.1 μM of each. 50 μl of blood from the patient was collected using the lancet. The blood was added to 150 μl extraction solution.

The vial was transported to the laboratory for analysis.

The vial was vortexed and centrifuged. 150 μl of the supernatant was collected and transferred to glass vials. The protein precipitation solution was evaporated to dryness and reconstituted in 60 μl 20% acetonitrile in water. 20 μl of the solution was injected into the LC-MS/MS system. The LC-MS/MS system: the mobile phases A; 0.1% formic acid in water and mobile phase B; 0.1% formic acid in acetonitrile.

Three validation batches were prepared and analyzed at three different occasions. Each validation batch comprised two sets of all calibration standards, two sets of all quality control samples and a number of blank samples.

The results show that there are no differences between a venous blood sample and a sample taken by a lancet in the finger.

### Example 3, Monitoring Amount of Methylmalonic Acid in Blood of a Patient

The kit comprised a vial that contained 100 μl extraction solution containing 0.5 μmol/L labelled MMA in water. 50 μl of blood from the patient was collected using the lancet. The blood was added to 100 μl extraction solution.

The vial was transported to the laboratory for analysis.

The vial was vortexed and transferred to a SPE column. The column is eluted by 150 μl 3% formic acid in acetonitrile. The eluate is evaporated to dryness. 100 μl water is added 20 μl is transferred to the LC-MS/MS system.

The mobile phases A: 0.05% acetic acid in 90% acetonitrile in water and mobile phase B: 0.5% formic acid in 20% acetonitrile in water.

The results show that there are no differences between a venous blood sample and a sample taken by a lancet in the finger

1. A method for measuring a concentration of one or more biomarker/analyte in blood from a mammal comprising:
providing a kit of parts for sampling blood, the kit of parts comprising a lancet and a capillary adapted to sample a defined volume of blood from the mammal and a vial comprising an extraction fluid together with one or more internal standard, wherein the one or more internal standard is a radioisotope of one or more biomarker selected from a group comprising one or more of 2H 3H, 11C, 13C, 14C, 13N, 15N, 15O, 17O and 18O radioisotope of said biomarker adapted to assess a quality and a concentration of the one or more biomarker in the blood sample,
distributing the kit of parts to a location of the mammal,
receiving the vial comprising a blood sample from the mammal inserted into the vial,
analysing the blood sample to determine the quality of the sample and the concentration of the one or more biomarker in the blood of the mammal by centrifuging the vial and performing a direct analysis of the supernatant, and normalizing the analyzed results between different samples and a standard curve.

2. The method according to claim 1, wherein the one or more internal standard is a 2H, 3H, 11C, 13C, 14C, radioisotope of one or more biomarker.

3. The method according to claim 1, wherein the one or more internal standard is a 13C and/or 2H radioisotope of one or more biomarker.

4. The method according to claim 1, wherein the one or more internal standard is a 2H radioisotope of one or more biomarker.

5. The method according to claim 1, wherein the one or more internal standard is a 13C radioisotope of one or more biomarker.

6. The method according to claim 1, wherein the one or more internal standard is adapted for measuring a quality and a concentration of one or more pharmaceutical active compound (API) that can be stabilized in an extraction fluid, whereby the API is selected from the group comprising antifungal API, antiviral API, immunodepression API, anticonvulsant API, antidepressant API, antibiotic API, anticancer API, vitamins, cardiovascular API, painkilling API and opiates.

7. The method according to claim 1, wherein the one or more internal standard is adapted for measuring or monitoring a quality and a concentration of one or more pharmaceutical active compound (API) in the blood of a mammal that can be stabilized in an extraction fluid, whereby the one or more API is selected from the group comprising tamoxifen, 4-hydroxytamoxifen, CPA-cyclophosphamide and its active metabolite PAM-hb, DOC-docetaxel, DOX-doxorubicine, PAC-paclitaxel, EPI-epirubicin, statins, steroids, such as testosterone, or vitamins, such as vitamin B or D, or compounds related to addictions, such as opioids, cocaine, heroin, fentanyl, cannabinoids, marijuana, benzodiazepines, hallucinogens and methamphetamine, codeine, ibuprofen, dihydrocodeine, morphine, dextropropxyphene napsylate, aminorex, lidocaine, iso-LSD, norcocaine, amitriptyline, pemoline, prazepam, imipramine, propafenone, pheniramine, chlorpromazine, amphetamine sulfate, lofexidine hydrochloride, clozapine, ecgonine methyl ester, diphenylhydramine, estazolam, 3,4-methylenedioxy-amphetamine, melatonin, doxepin, ketamine, mescaline, aprobarbital, buprenorphine, benzoylecgnine, trifluoperazine, methadone, ecgonine ethyl ester, midazolam, fentanyl, norketamine, chlordiazepoxide, caffeine, hydrocodone, fenfluramine, tramadol, lorazepam, phenylpropanolamine, flunitrazepam, 2C—B, amobarbital, flurazepam, phencyclidine (PCP), barbital, carbamazepine, vancomycin and phenobarbital.

8. The method according to claim 1, wherein the one or more internal standard is a radioisotope of tamoxifen, z-endoxifen and/or 4-hydroxytamoxifen adapted for measuring the amount of tamoxifen, z-endoxifen and/or 4-hydroxytamoxifen in the blood of a mammal.

9. The method according to claim 8, wherein the internal standard is a 13C-radioisotope of tamoxifen, z-endoxifen and 4-hydroxytamoxifen.

10. The method according to claim 1, wherein the one or more internal standard is phosphatidylethanol 16:0/18:1 (PEth-16:0/18:1, PEth 16:0/18:2, PEth 18:1/16:0, and/or PEth 18:2/16:0) adapted for measuring long-term alcohol consumption.

11. The method according to claim 1, wherein the one or more internal standard is a radioisotope of methylmalonic acid adapted for measuring B-vitamin deficiency in a mammal.

12. The method according to claim 1, wherein the one or more internal standard is a radioisotope of dihydrotachysterol adapted for measuring D-vitamin deficiency in a mammal.

13. The method according to claim 1, wherein the one or more internal standard is adapted for measuring or monitoring a quality and a concentration of one or more pharmaceutical active compound (API) in the blood of a mammal that can be stabilized in an extraction fluid, whereby the one or more API is selected from the group comprising compounds related to addictions, such as opioids, cocaine, heroin, fentanyl, cannabinoids, marijuana, benzodiazepines, hallucinogens and methamphetamine, amphetamine, codeine, dihydrocodeine, morphine, iso-LSD, phencyclidine (PCP), norcocaine, fentanyl, methadone, hydrocodone and tramadol.

14. The method according to claim 1, wherein the one or more internal standard is adapted for measuring or monitoring a quality and a concentration of one or more statins selected from the group comprising atorvastatin, fluvastatin, cerivastatin, lovastatin, mevastatin, pitavastatin, pravastatin, rosuvastatin and simvastatin, or mixtures thereof.

15. The method according to claim 1, wherein the one or more internal standard is adapted for measuring or monitoring a quality and a concentration of one or more steroid hormones selected from the group comprising alclometasone, prednisone, dexamethasone, triamcinolone, cortisone, fludrocortisone, oxandrolone, oxabolone, testosterone, nandrolone, diethylstilbestrol (DES) and estradiol, norethisterone, medroxyprogesterone acetate, hydroxyprogesterone caproate, cyproterone acetate, mifepristone and gestrinone, or mixtures thereof.

16. The method according to claim 1, wherein the one or more internal standard is adapted for measuring or monitoring a quality and a concentration of one or more antidepressant API selected from the group comprising bupropiontrazodone, nefazodone, vilazodone, vortioxetine, mitriptyline, bupropion, bupropion, bupropion, bupropion, bupropion, citalopram, desvenlafaxine, duloxetine, escitalopram, fluoxetine, mirtazapine, nortriptyline, paroxetine, sertraline, trazodone, venlafaxine, or mixtures thereof.

17. The method according to claim 1, wherein the blood sample is analysed using mass spectrometry (MS), liquid chromatography-mass spectrometry (LC-MS, LC-MS/MS, gas chromatography-mass spectrometry (GC-MS) or GC-MS/MS.

18. The method according to claim 1, wherein the extraction fluid is selected from the group comprising 2-propanol, methanol and acetonitrile, formic acid or mixtures thereof, which fluid is adapted to substantially stop all enzyme activity in the blood sample, stabilize the sample and extract the biomarker from the blood sample.

19. The method according to claim 1 for use in measuring or monitoring a quality and a concentration of one or more active pharmaceutical compound in the blood of a mammal, whereby the pharmaceutical active compound has a narrow therapeutic window selected from the group comprising or consisting of tamoxefin, digoxin, digitoxin, fosphenytoin, phenytoin, ethosuximide, alfentanil, tacrolimus, meperidine, temsirolimus, sirolimus, thiopental, fentanyl, alfentanil, theophylline, cyclosporine, clonidine, amitriptyline, protriptyline, imipramine, nortriptyline, quinidine, levothyroxine, carbamazepine, phenobarbital, ergotamine, dihydroergotamine and heparin.

20. The method according to claim 1, for use in Tailor Dose Monitoring (TDM).

21-22. (canceled)
